(19) United States
(12) Patent Application Publication
(10) Pub. No.: US 2019/0201646 A1
(43) Pub. Date: Jul. 4, 2019

(54) RESPIRATORY ASSISTANCE DEVICE AND SYSTEM AND METHOD

(71) Applicant: BIDIBOTS PTY LTD, Reservoir (AU)

(72) Inventor: Mark GILLIES, Enmore (AU)

(21) Appl. No.: 16/222,134

(22) Filed: Dec. 17, 2018

Related U.S. Application Data
(63) Continuation of application No. 13/130,694, filed on May 23, 2011.

(30) Foreign Application Priority Data
Nov. 21, 2008 (AU) 2008906036

(51) Int. Cl. A61M 16/06

(52) U.S. Cl. CPC A61M 16/0666

(57) ABSTRACT

A pressurized air delivery device, system and method provide respiratory assistance to individuals, particularly infant humans. The device having nasal conduits or prongs which are in fluid communication with respective supply conduits separately supply each nasal conduit with a breathable gas such as air or oxygen. The size of the nasal conduits or prongs may be chosen according to the size of the individual's nares and the spacing between nasal conduits or prongs may be adjustable.

### CROSS-REFERENCE TO RELATED APPLICATIONS

This application is a continuation of U.S. application Ser. No. 13/130,694 filed May 23, 2011, which is the U.S. national phase of PCT application No. PCT/AU2009/001519 filed Nov. 23, 2009, which claims priority to Australian application 2008906036 filed Nov. 21, 2008, the disclosure of which is incorporated in its entirety by reference herein.

### FIELD OF THE INVENTION

This disclosure relates to delivery of continuous positive airway pressure to an individual, such as an infant, to thereby provide respiratory assistance.

### BACKGROUND

Newborn, infants particularly those born premature often have a need for respiratory support, ranging from increased ambient oxygen, through continuous positive airways pressure (CPAP) and endotracheal ventilation to, rarely, extracorporeal membrane oxygenation (ECMO). Infants born before 30 weeks of completed gestation almost invariably need a period of CPAP or ventilation to survive.

The commonest reason for premature newborn infants to require respiratory support is due to a lack of surfactant, a protein which helps to keep the smallest of the lungs air spaces open. Treatment for this disease process has consisted of CPAP since it was realised in the mid 1970's that CPAP treatment greatly improved infants' rates of survival.

Whilst replacement of surfactant via an endotracheal tube has been available in Australia since the early 1990's, this requires infants to be intubated and ventilated. However intubation and ventilation, itself, can damage infants' lungs and there has been a shift in the last 5-10 years towards CPAP, both as a modality of support post extubation and a primary support in its own right, for the majority of infants with respiratory distress.

CPAP may be delivered via a multitude of machine patient interfaces. The best devices utilise “short binasal” prongs designed to snugly fit into the nares of infants, thereby allowing the pressure to be delivered to the infant whilst minimising the resistance of the device to the infants' work of breathing.

Current “short binasal” prongs are designed with a connection between the two prongs which insert into the infant's nose. This connection may cause pressure on the nasal septum, which in extreme cases can lead to necrosis and require plastic surgery to correct. This problem is exacerbated by the mechanism by which these prongs are fixed to the infant's head and face by means of hats, bonnets or headbands and the relatively bulky tubing and prongs in use.

Although the currently available devices come in a variety of sizes, both for prong size and separation between the prongs, each prong set is typically of a fixed prong size and separation.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### SUMMARY

The invention is broadly directed to providing respiratory assistance to an individual, typically an infant, by way of a device, system and/or method that delivers pressurized air to the nares of the individual, to alleviate one or more of the aforementioned deficiencies of the prior art.

In one aspect, the invention provides a pressurized air delivery device for providing respiratory assistance to an individual, said device comprising a plurality of nasal conduits in fluid communication with respective supply conduits connectable to a source of pressurized, breathable gas.

In another aspect, the invention provides a pressurized air delivery system for providing respiratory assistance to an individual, said system comprising a plurality of flexible nasal conduits in fluid communication with respective supply conduits connected to a source of pressurized, breathable gas.

In yet another aspect, the invention provides a method of providing respiratory assistance to an individual, said method including the step of delivering pressurized air to the nose of said individual through a plurality of nasal conduits in fluid communication with respective supply conduits connected to a source of pressurized, breathable gas.

Preferably, according to the aforementioned aspects, the individual is an infant human.

Suitably, continuous positive airway pressure is delivered to an individual by way of a plurality of conduits respectively locatable in the nares of an individual, without engaging, bearing against or otherwise contacting the nasal septum. Advantageously, continuous positive airway pressure is delivered to the individual without providing a hat, bonnet, headband or other separate means for attachment to the individual.

In a preferred embodiment, the nasal conduits are of a size adapted to fit the nares of the individual. In a particularly preferred embodiment the nasal conduits are interchangeable in size.

Preferably the nasal conduits are formed of a material that is more rigid than the supply conduits.

In certain embodiments, the nasal conduits may each comprise an inlet and an outlet, said outlet facilitating exhaust of exhaled gas to atmosphere.

Preferably, the nasal conduits further comprise respective valves to control oxygen flow to the user.

Suitably, the device and/or system further comprises a spacer to maintain a desired spacing or distance between the nasal conduits.

Preferably, the spacer is adjustable in order to select a desired spacing or distance between the nasal conduits.

In one preferred embodiment, said respective nasal prongs are maintained at a desired distance apart by an adjustable spacer that comprises a sliding rod or a band that comprises teeth or serrations. Typically, the adjustable spacer comprises an adjustment member, such as a lever, ratchet, turnbuckle, or cam, although without limitation thereto.

In a particularly preferred embodiment, the spaces comprises a band comprising teeth, ridges or serrations releasably engageable by a cam or lever.

Throughout this specification, unless the context requires otherwise, the words “comprise”, “comprises” and “comprising” will be understood to imply the inclusion of a stated integer or group of integers but not the exclusion of any other integer or group of integers.

### BRIEF DESCRIPTION OF THE DRAWINGS

A preferred embodiment of the invention will now be described by way of example only, with reference to the accompanying figures in which:

FIG. 1 shows an embodiment where nasal prongs being held at a desired distance apart by rare earth magnets;

FIG. 2A shows an embodiment where nasal prongs are held at a desired distance apart by a ratchet mechanism;

FIG. 2B shows a detail view of the ratchet mechanism;

FIG. 3A shows an embodiment where nasal prongs are held at a desired distance apart by a turnbuckle system;

FIG. 3B shows a side view of the embodiment of FIG. 3A disposed in a nose;

FIG. 4A shows an embodiment where nasal prongs are spaced apart by a sliding rod mechanism;

FIG. 4B shows a side view of the embodiment of FIG. 4A disposed in a nose;

FIG. 4C shows a detail view of a push button of the embodiment of FIG. 4A in a closed position;

FIG. 4D shows a detail view of the push button of the embodiment of FIG. 4A in an open position;

FIG. 5A shows a front view of nasal prongs according to another embodiment;

FIG. 5B shows the embodiment of FIG. 5A disposed in a nose;

FIG. 5C shows a top view of the nasal prongs of FIG. 5A;

FIG. 5D shows a detail view of a push button of the embodiment of FIG. 5A in a closed position;

FIG. 5E shows a detail view of the push button of the embodiment of FIG. 5A in an open position;

FIG. 6A shows a top view of nasal prongs spaced apart by a sliding locking flat band mechanism;

FIG. 6B shows a front view of the nasal prongs of FIG. 6A;

FIG. 6C shows a detail view of the sliding locking flat band of FIG. 6A;

FIG. 6D is a cross-sectional view of the sliding locking flat band of FIG. 6C;

FIG. 7A shows nasal prongs spaced apart by a sliding locking flat band mechanism that is adjustable by a cam mechanism;

FIG. 7B shows a detail view of the sliding locking flat band of FIG. 7A;

FIG. 7C shows a cross-sectional view of the sliding locking flat band of FIG. 7B;

FIG. 8 shows an example of the attachment of nasal prongs to an air supply conduit;

FIG. 9A shows a nasal prong comprising and inlet and outlet for exhalation;

FIG. 9B shows a cross-sectional view of the nasal prong of FIG. 9A;

FIG. 9C shows a detail view of the inlet of the nasal prong of FIG. 9A;

FIG. 9D shows a detail view of the outlet of the nasal prong of FIG. 9A; and

FIG. 9E shows a screen of the nasal prong of FIG. 9A.

### DETAILED DESCRIPTION

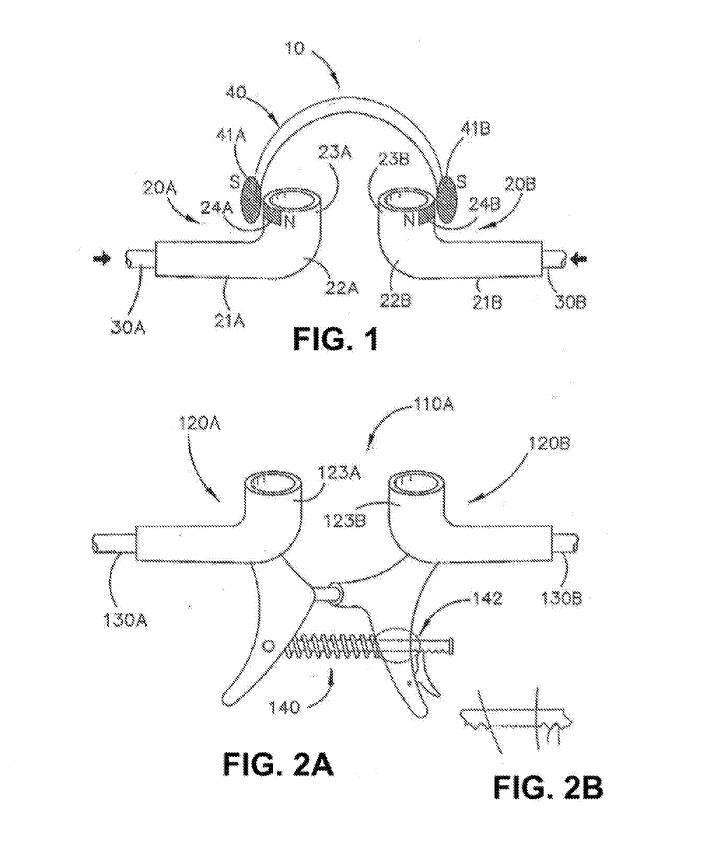

Referring to FIG. 1, device 10 comprises respective nasal conduits 20A, 20B in fluid communication with respective air and/or oxygen supply conduits 30A, 30B. Nasal conduits 20A, 20B respectively comprise arms 21A, 21B normal to air supply conduits 30A, 30B and elbows 22A, 22B which respectively terminate in nasal prongs 23A, 23B. Preferably nasal conduits 20A, 20B are formed of a material that is more rigid (e.g. polymethylmethacrylate or polycarbonate) than the supply conduits which are of a relatively flexible tubing material (e.g. polyurethane).

In use, air and/or oxygen supply conduits 30A, 30B are connected to a source of pressurized air, oxygen or other gas suitable for assisting human respiration (not shown). The direction of pressurized air flow from the source is indicated by solid arrows.

According to this embodiment, spacer 40 comprises magnetic poles 41A, 41B on bridge 42, which respectively interact with magnetic poles 24A, 24B on nasal prongs 23A, 23B to maintain nasal prongs 23A, 23B at a desired separation. Typically, although not exclusively, magnetic poles 41A, 41B, 24A and 24B are produced by rare earth magnets (e.g. lanthanides such as neodymium or samarium, although without limitation thereto) located on the outside of the nares.

Referring to an embodiment shown in FIGS. 2A and 2B, device 110 comprises spacer 140 comprising adjustable ratchet 142 which maintains nasal prongs 123A, 123B at a desired separation.

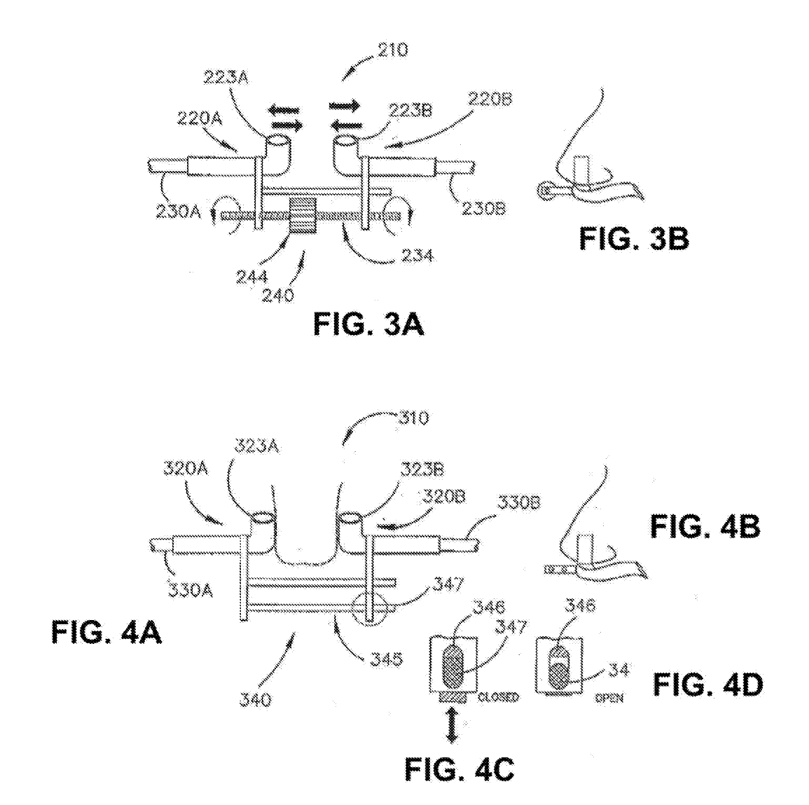

An embodiment shown in FIGS. 3A and 3B shows nasal prongs 223A, 223B adjustably movable (indicated by solid horizontal arrows) to a desired distance apart by spacer 240 comprising turnbuckle 243 that is adjustable by winding wheel 244 in either direction (indicated by solid arrows).

In another embodiment of device 310 shown in FIGS. 4A through 4D, nasal prongs 323A, 323B are held at a desired distance apart by spacer 340 that is adjustable by way of sliding rod mechanism 345 that is adjustable by pushing button 346 to release sliding rod 347 and thereby finding the correct distance between nasal prongs 323A, 323B.

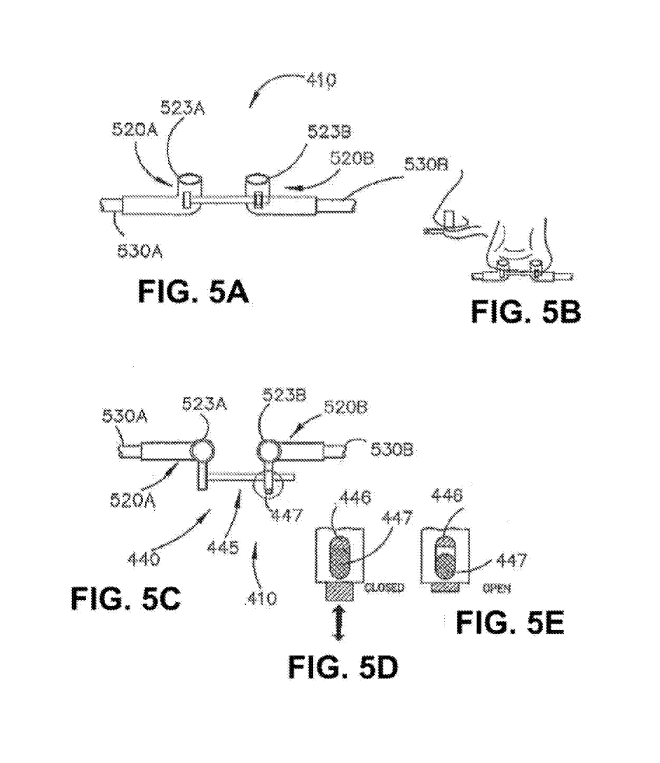

Referring now to FIG. 5A through 5E, an embodiment of device 410 is shown similar to that shown in FIG. 4A, wherein nasal prongs 423A, 423B are of reduced diameter compared to those in FIG. 4A, demonstrating that nasal prongs 23A, 23B may be of varying, interchangeable sizes.

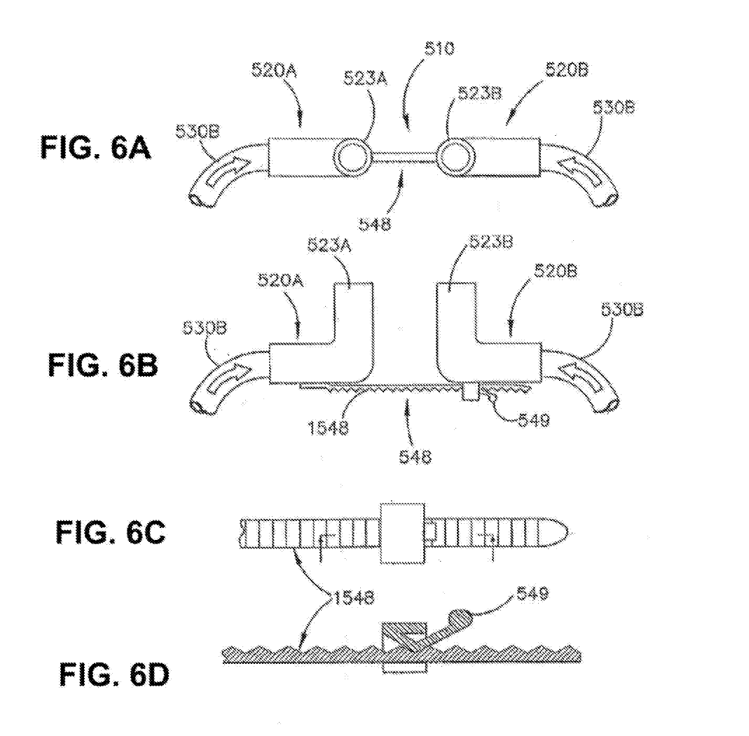

FIGS. 6A through 6D shows an embodiment where nasal prongs 523A, 523B are held at a desired distance apart by spacer 540 comprising slidable band 548 that comprises teeth, serrations or ridges 1548 releasably engageable by lever 549. Lever 549 engages teeth, serrations or ridges 1548 of flat band 548 to releasably maintain the desired distance between prongs 523A, 523B and thereby allow adjustment of the spacing between prongs 523A, 523B. Preferably, slidable band 548 is located underneath arms 521A, 521B of nasal conduits 520A, 520B (relative to an individual's nares) to maximize the distance between slidable band 548 and the individual's nasal septum. This should eliminate or at least minimize any potential contact between slidable flat band 548 and the individual's nasal septum.

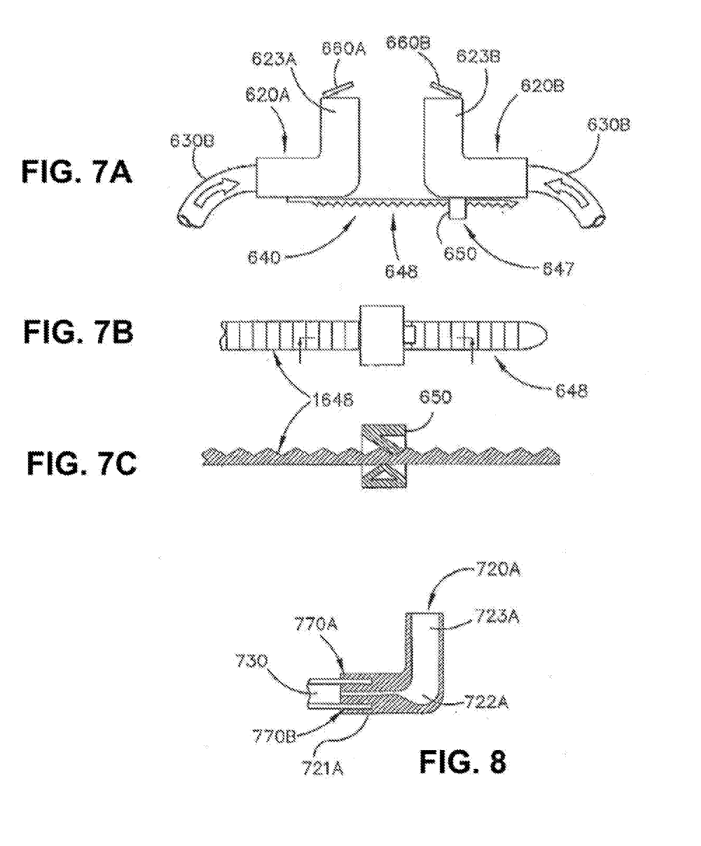

In an embodiment shown in FIGS. 7A though 7C, nasal prongs 623A, 623B are held at an appropriate distance apart by spacer 640 comprising slidable t band 648 that comprises teeth, serrations or ridges 1648 releasably engageable by cam 650. Cam 650 releasably engages teeth, serrations or ridges 1648 of slidable band 648 to releasably maintain the desired distance between prongs 623A, 623B and thereby allow adjustment of the spacing between prongs 623A, 623B. In this embodiment, slidable band 648 is ideally located underneath arms 621A, 621B of nasal conduits 620A, 620B (relative to an individual's nares) to maximize the distance between slidable band 648 and the individual's nasal septum.

It should also be appreciated that slidable band 540, 640 is shown as a “flat” band in FIGS. 6 and 7. However, in certain embodiments slidable band 540, 640 may be curved to facilitate a more comfortable fit on the individual.

FIG. 8 shows an example of one way in which nasal conduit 720A is attached to air supply conduit 730A. In this example, arm 721A is pushed onto air supply conduit 730A and barbs 770A, 770B are employed to hold arm 721A in place on air supply conduit 730A.

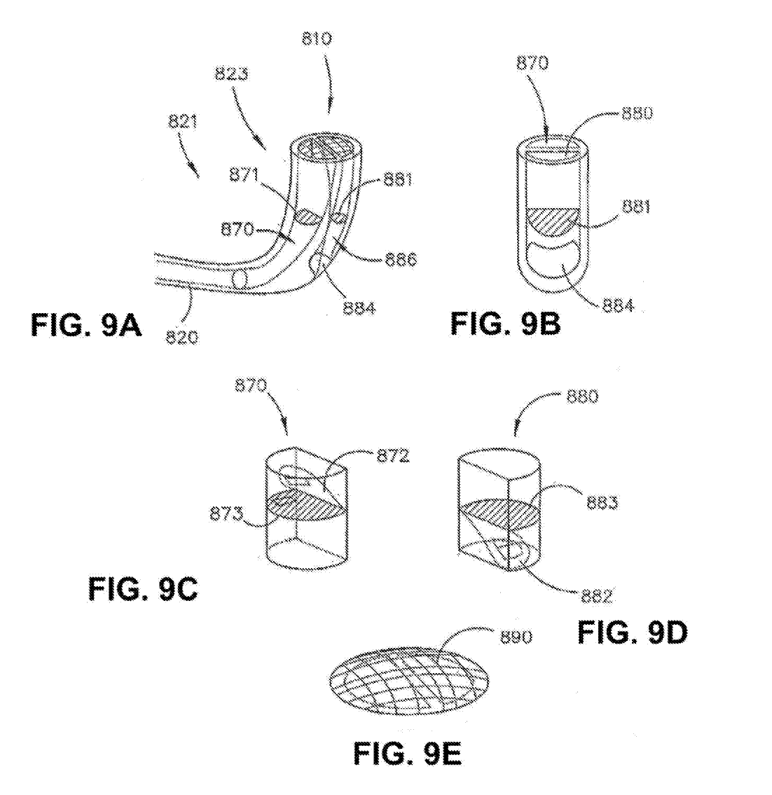

Referring to FIGS. 9A through 9E, there is shown an embodiment wherein device 810 comprises nasal prong 823 having inlet 870 and outlet 880, which each operate as “one-way” valves. Inlet 870 comprises leaflet 871 which moves between open position 872 and closed position 873 to enable a one-way flow of air to the nares. Outlet 880 comprises leaflet 881 which moves between closed position 882 and open position 883 to enable a one-way flow of exhaled gas from the nares to atmosphere via aperture 884. Screen 890 may also be provided to prevent accidental inhalation of leaflets 871 and/or 881.

It will be appreciated the present invention provides continuous positive airway pressure to an individual, typically an infant human, without engaging or otherwise contacting the nasal septum in a manner that causes irritation or discomfort to the user. Air, oxygen or other breathable gas is supplied via separate air supply conduits and nasal conduits, rather than via a common air supply conduit into separate nasal conduits. This eliminates the portion of the common air supply conduit that contacts and irritates the nasal septum. Furthermore, the nasal prongs may be provided in a variety of interchangeably different sizes to suit the size of the nares of the user, which also assists the objective of reducing the level of irritation and discomfort.

Another advantage of the present invention is that the device needs limited fixation, (even just a piece of tape may suffice) rather than the bonnets, hats, head-bands etc. required with prior art devices. Furthermore, many prior art devices require significant nursing expertise to attach and supervise in use. The present disclosure can be fitted and supervised by any person familiar with standard nasal cannulae.

In summary, the present invention provides improved nasal septal protection, ease of attachment, ease of nursing care, reduced weight and bulk, the ability to adapt nasal conduit size to different nare sizes (sometimes if a child has been nasally intubated, one nare is larger than the other), and variable separation between respective conduits.

Throughout this specification, the aim has been to describe the preferred embodiments of the invention without limiting the invention to any one embodiment or specific collection of features. Various changes and modifications may be made to the embodiments described and illustrated without departing from the present invention.

What is claimed is:

1. A pressurized air delivery device for providing respiratory assistance to an infant human, said device comprising:
a pair of nasal conduits each having a nasal prong for insertion into a nostril of an infant and a hollow, rigid arm oriented at approximately 90° to the nasal prong forming an elbow shape;
an adjustable spacer for assembly including a first portion and a second portion each connected to a different one of the hollow, rigid arms, wherein the adjustable spacer maintains the nasal prongs in spaced apart parallel orientation with the arms extending in an opposed outward orientation aligned along a common transverse axis, the first and second portions adjustably cooperating with one another to enable the spacing between the nasal prongs to be varied to fit the infant; and
a pair of supply conduits having ends in fluid communication with the outwardly oriented ends of the pair of nasal conduits, the supply conduits having opposed ends connectable to a source of pressurized breathable gas,
wherein, in use, the nasal conduits are located in the nostrils of the infant human to deliver continuous positive airway pressure without engaging or otherwise contacting the nasal septum.

2. The device of claim 1, wherein the adjustable spacer is selected from a group consisting of a lever, ratchet and a cam.

3. The device of claim 1, wherein the nasal conduits are removably attached to the supply conduits and of interchangeable sizes.

4. The device, of claim 1, wherein the nasal conduits each further comprise a valve.

5. The device of claim 1, wherein each said nasal conduit comprises an inlet for flow of air into nares of a user and an outlet, said outlet facilitating exhaust of exhaled gas to atmosphere.

6. The device of claim 1, wherein the breathable gas is air or oxygen.

7. A method of providing respiratory assistance to an infant human, said method including the steps of providing a pressurized air delivery device according to claim 1 and delivering pressurized air to a nose of said infant human through the nasal conduits.

8. The device of claim 1, wherein portions of the nasal conduits which are to be received by the infant human extend in a first direction and the spacer is disposed on a side of the device which is generally opposite from the first direction.
